(19) United States
(12) Patent Application Publication
(10) Pub. No.: US 2008/0085836 A1
(43) Pub. Date: Apr. 10, 2008

(54) Method for genetic testing of human embryos for chromosome abnormalities, segregating genetic disorders with or without a known mutation and mitochondrial disorders following in vitro fertilization (IVF), embryo culture and embryo biopsy

(76) Inventors: William G. Kearns, Poolesville, MD (US); Richard A. Leach, Spring Lake, MI (US)

Correspondence Address: Ober, Kaler, Grimes & Shiver;Royal W. Craig, 120 East Baltimore Street, Baltimore, MD, 21202-1643, US

(21) Appl. No.: 11/903,587

(22) Filed: Sep. 24, 2007

Related U.S. Application Data
(60) Provisional application No. 60/846,801, filed on Sep. 22, 2006.

(51) Int. Cl. C40B 20/00; C12P 19/34; C40B 40/06

(52) U.S. Cl. 506/2; 435/91.2; 506/16

(57) ABSTRACT

We describe a method for interrogating the content and primary structure of DNA by microarray analyses and to provide comprehensive genetic screening and diagnostics prior to embryo transfer within an IVF setting. We will accomplish this by the following claims: 1) an optimized embryo grading system, 2) a less invasive embryo biopsy with reduced cellular contamination, 3) an optimized DNA amplification protocol for single cells, 4) identify aneuploidy and structural chromosome abnormalities using microarrays, 5) identifying sub-telomeric chromosome rearrangements, 6) a modified DNA fingerprinting protocol, 7) determine imprinting and epigenetic changes in developing embryos, 8) performing genome-wide scans to clarify/diagnose multi-factorial genetic disease and to determine genotype/haplotype patterns that may predict future disease, 9) determining single gene disorders with or without a known DNA mutation, 10) determining mtDNA mutations and/or the combination of mtDNA and genomic (nuclear) DNA aberrations that cause genetic disease.

### BACKGROUND OF THE INVENTION

1. Field of the Invention

The present invention relates to a method for genetic testing of human embryos and, more particularly, to a method for optimized whole genome amplification using an enhanced multiple displacement amplification protocol and a modified microarray platform for preimplantation genetic diagnosis (PGD) and screening following IVF, embryo culture and embryo biopsy.

2. Description of the Background

There is a variety of existing commercial and humanitarian needs for in vitro fertilization (IVF). Genetic issues play a significant role in a couple's ability to achieve a viable pregnancy and the birth of a normal baby. These genetic issues include numerical chromosome abnormalities (aneuploidy), structural chromosome aberrations (translocations, inversions, duplications and/or deletions), single gene disorders (fragile X, etc) and mitochondrial abnormalities (Keams-Sayre syndrome, etc). IVF and genetic testing are described as follows:

IVF-Genetic Testing (PGD)

IVF is a very successful treatment for infertility and is a required procedure for couples in need of Preimplantation Genetic Diagnosis and screening. Every couple attempting this type of therapy should understand the nuances of the treatment and be realistic about their chance of success. Careful evaluation and diagnosis of the infertility problem, full pre-screening, and an individualized approach to treatment will maximize each couple's chances for success.

An enhanced and optimized IVF treatment protocol including ovulation induction, oocyte (egg) retrieval, oocyte fertilization, specialized embryo culture protocols and embryo transfer techniques along with a specialized and enhanced embryo biopsy and PGD testing will optimize couples' chances of achieving a normal pregnancy and delivering a healthy baby.

Step 1—Ovulation Induction

Women undergoing IVF require hormone injections to stimulate follicular development and multiple egg production. This stimulation process usually requires the initial use of a gonadotropin releasing hormone (GnRH) agonist to suppress ovarian function, preventing ovulation until the desired time. A protocol individualized for particular hormone levels and history should be employed. Daily injections of gonadotropins are then added to stimulate the development of follicles and eggs. These are usually given subcutaneously (under the skin). The progress of ovulation induction is then monitored with ultrasounds and blood hormone levels over several days.

Step 2—Egg Retrieval

Egg retrieval involves placing a special needle into the ovarian follicle and removing the fluid that contains the egg. This is a relatively minor procedure and is performed by visualizing the follicles with a vaginal ultrasound probe. A needle is directed alongside the probe, through the vaginal wall, and into the ovary. To avoid any discomfort to the patient, a short acting intravenous sedation is provided.

Step 3—Fertilization, Embryo Culture and PGD

Once the follicular fluid is removed from the follicle, the microscopic eggs are identified by the embryologist and placed into an incubator. Conventional insemination or intracytoplasmic sperm injection (ICSI) is used to fertilize the eggs. The type of fertilization employed is based on the male's semen parameters and/or the type of PGD required. ICSI is required for all PGD testing employing microarray analysis or DNA sequencing but not PGD testing using fluorescence in situ hybridization.

During “conventional” insemination approximately 50,000 sperm are mixed with each egg in a culture dish and incubated overnight to undergo the fertilization process. Intracytoplasmic sperm injection is a technique whereby one sperm is directly injected into one egg. With either fertilization technique, the eggs are checked the next day to document fertilization and the day after to evaluate for early cell division. The fertilized eggs are now called embryos and are placed in a special culture media to promote growth and development. If embryo biopsy and PGD is scheduled, it is at this point (day-3 of development) that one or two blastomeres are removed from each cleaving embryo by a procedure called Embryo Biopsy and transferred to the Preimplantation Genetics Laboratory for genetic testing.

The ability to grow an embryo in culture and to optimize embryo growth and development is a difficult technique to master. Many IVF labs only grow embryos to day-3 and then transfer chosen embryos to the patient's uterus and/or cryopreserve (freeze) for future use. Enhanced embryology techniques have given some laboratories the ability to grow the embryos for five, six or seven days in culture until they reach the blastocyst stage. These blastocysts may have a greater chance of implantation, and allow IVF clinics to transfer fewer embryos, reducing the risk of multiple births while increasing the chance of a viable clinical pregnancy.

Day-3 biopsied and PGD genetically diagnosed embryos are transferred on day 5, 6 or 7 following egg retrieval. They are placed through the cervix into the uterine cavity using a small, soft catheter. This procedure usually requires no anesthesia.

Embryo Grading

Embryo grading (assessment) plays a significant role in the identification of “the best” morphological embryo, which is the one most likely to implant and to achieve a viable pregnancy. Standardized embryo grading systems have been developed but this varies from lab to lab. Some problems encountered with embryo assessment include its subjective grading scale, poor standardization, its time of evaluation (i.e. what time of day are the embryos evaluated), and the removal of embryos from the incubator for microscopic review, which can compromise embryo development and pre and post genomic activation. Studies on grading systems between laboratories and embryologists scoring embryos have shown that interobserver variability and intraobserver variability among embryologists are significant. This variability could alter both the experimental adjustments required to optimize embryo growth and development as well as choosing the “best” embryo for transfer, both of which directly impact IVF pregnancy rates and IVF program success. An enhanced embryo grading system to identify the “best embryo” for successful implantation and the birth of a live baby is required to increase IVF success rates.

Embryo Biopsy

In almost all cases, embryo biopsy is the removal of a blastomere from a day-3 embryo so as to complete PGD for chromosome disorders, segregating single gene mutations and/or mitochondrial disorders. In rare cases, a day-5, 6 or 7 biopsy is done on trophectoderm cells of a blastocyst for segregating genetic disorders. Three basic biopsy techniques exist and these vary from lab to lab. These techniques include laser, acid tyrodes and mechanical. All three have significant risks of damaging a day-3 embryo during biopsy with a subsequent reduction in implantation rates, a possible increase in biochemical pregnancies and a reduction in the birth of healthy, normal babies. One recent scientific publication from the New England Journal of Medicine suggested that day-3 embryo biopsies induce significant damage to the in vitro developing embryo and reduces implantation by approximately 30%. Others disagree with this risk. Therefore, we suggest that an enhanced and optimized embryo biopsy technique is required so as to reduce the risk of significantly damaging the embryo during the biopsy procedure.

Preimplantation Genetic Diagnosis and Screening (PGD)

Cells contain chromosomes, which are string-like structures where all of our genetic material resides. The genetic material is called a gene. Genes are made up of DNA sequences. Each cell has approximately 25,000 genes. Cells also contain mitochondrial organelles that contain a different type of DNA. Genetic disease is caused by abnormalities of gene function. This can occur by having too many or too few chromosomes (aneuploidy), when chromosome pieces are attached to the wrong chromosome (translocation), when one is missing or containing an extra piece of a chromosome (deletion or duplication), when part of a chromosome is upside down (inversion), or when the genomic (nuclear) or mitochondrial DNA sequence is changed.

Currently, one can test embryos for chromosome disorders, single gene mutations and some mitochondrial disorders.

Fluorescence in situ hybridization (FISH) employs DNA probes for a single locus on individual chromosomes. One visualizes the fluorescent signal within an embryo blastomere or trophectoderm interphase nucleus using a special fluorescent microscope. This technique works well but is limited by the fact that one cannot simultaneously analyze all 23 pairs of human chromosomes. Current PGD testing analyzes approximately 5-12 chromosomes and misses abnormalities of the remaining undiagnosed chromosomes. This can lead to the transfer of a genetically abnormal embryo. FISH also only targets one specific locus of specific DNA. Altered chromosome condensation or nuclear membrane hardness can falsely infer a nucleus as missing a chromosome(s) when in fact, it's a failure of probe to reach and hybridize to the target DNA. Another problem with FISH is that most probes target repetitive or satellite DNA sequences. This DNA is made up of base pair repeats and varies slightly from centromere to centromere of different chromosomes. This can lead to cross-hybridization to non-target chromosomes and incorrectly diagnosing an embryo as having additional copies of that chromosome and calling the embryo genetically abnormal.

Single gene abnormalities (mutations) are caused by nucleotide changes in the sequence of genomic (nuclear) DNA. Genes produce proteins that make our cells work properly. Single gene disorders usually show a characteristic family history of a specific genetic disease. Mitochondrial mutations can also cause disease and segregate in families. DNA mutations can alter the cells' normal function due to a lack of a required protein. For example, Cystic Fibrosis (CF) is a common genetic disorder that primarily affects the lungs of CF patients. The CF mutation affects a protein within the cell that reduces the cells ability to function properly. This results in a build up of mucous within the lungs, lung dysfunction and possible death. Mitochondrial disorders are a clinically heterogeneous group of disorders that arise as a result of dysfunction of the mitochondrial metabolic chain. They can be caused by mutations of genomic (nuclear) or mitochondrial DNA (mtDNA). Some mitochondrial diseases are due to an interaction between gene products of mtDNA and genomic (nuclear) DNA mutations. Due to the complexity of mtDNA and the rare instances where the sequence is known that causes the disease, few PGD cases are done for mitochondrial disorders.

The current state of genomic (nuclear) genetic testing on blastomeres or trophectoderm cells includes polymerase chain reaction (PCR), DNA sequencing and/or linkage analysis. Only genetic diseases with a known gene mutation or where a large family with a segregating genetic disease always associated with a known linked genetic marker can undergo PGD testing. Limitations include the potential mis-diagnosis due to allele drop-out (missing genetic material) of one partners' DNA, preferential DNA amplification, laboratory errors due to inaccurate primer design for the PCR reaction, PCR and/or sequencing failure, laboratory contamination during the biopsy procedure (i.e. operator induced DNA contamination from an exogenous source) or the inability to test only for segregating genetic disorders with a known DNA mutation.

A significant limitation in PGD testing is the inability to simultaneously diagnose chromosome, single gene, mitochondrial and diseases segregating without a known mutation from a single cell. This is due to differences in current experimental methodology. Chromosome based methods require FISH on an intact interphase nucleus of a single cell, whereas the others need extracted and amplified genomic (nuclear) and/or mtDNA.

Another significant issue with current PGD technology is a mis-diagnosis. Overall, the reported world-wide mis-diagnosis rate is at least 6-11%. A mis-diagnosis can result in a spontaneous miscarriage or the birth of a child with a genetic disorder. It is clear that more robust methodologies are required for PGD.

Polymerase Chain Reaction

As briefly discussed above, PCR is used in PGD testing and is a standard molecular biology procedure that was developed in the mid 1980's for the purpose of amplifying defined segments of genetic material and is used routinely in both research and medical diagnostic laboratories.

Briefly, this methodology exploits the physical properties of the naturally occurring DNA polymerase from the thermophilic bacteria Thermophylis aquaticus (Taq) to remain functional at high temperatures. This Taq polymerase is used in an iterative process of DNA replication in vitro. PCR is typically considered to have three steps to the process: 1) DNA denaturation, 2) primer annealing, and 3) chain elongation. For DNA denaturation, the assay temperature is brought to ˜95° C. to disrupt the hydrogen bonds between the nitrogenous bases of the nucleic acid secondary structure. Once denatured, the assay reaction temperature is reduced to a temperature that is sufficiently low enough for short, sequence-specific oligonucleotides to hybridize to the denatured genomic DNA recreating a local 2° structure, usually ˜62° C. During chain elongation, the reaction temperature is raised to ˜72° C., the optimal temperature for Taq polymerase, and the hybridized primer is extended as a function of polymerase fidelity. These three steps are repeated in an iterative, programmatic assay controlled by a thermocycler. PCR is a robust technique but can preferentially amplify or fail to amplify genomic DNA. Therefore, it should not be used for DNA amplification from single cells.

Multiple Displacement Amplification

Whereas PCR is useful for amplifying short genomic segments (approximately <5 kb), another type of DNA amplification, multiple displacement amplification, is more useful for replicating the entire genome in vitro. Multiple Displacement Amplification (MDA) was developed in the 1990's and is now one experimental method for replicating the entire genome of an organism. MDA exploits the replication fidelity of the bacterial DNA polymerase Φ29. Φ29 can flawlessly replicate DNA with less than one error every 1×10⁶ bases. MDA is beginning to be employed in genetics research to increase the quantity of substrate DNA required for high-density genomic analysis.

The standard MDA assay is quite simple. In its typical use, nanogram quantities of DNA is obtained from several to dozens of cells within a homogeneous cell population and is used in various types of genetic experimental protocols. Basically, substrate DNA is mixed into a reaction cocktail with Φ29 and random oligonucleotide primers and incubated at constant temperature for several hours. At ˜25° C., Φ29 has tremendous ability to displace DNA 2° structure without the intervention of mammalian helicases. Rapid, high fidelity DNA replication allows for a linear, unbiased genome amplification that copies >99.8% of the substrate genome. Moreover, half-milligram quantities of DNA can be amplified from small groups of cells. However, no optimized experimental protocol currently exists for the successful isolation, amplification and application of MDA prepared DNA from day-3 or trophectoderm single cells for preimplantation genetic diagnosis and screening.

Genotyping Microarrays for Whole Genome Analysis

The advent of microarray technology shifted the paradigm of genomic research from specific hypothesis driven research to broad-scale data driven research. Quite simply, the microarray, regardless of its incarnations, provides a platform upon which millions of individual assays may be performed simultaneously. The massive data set that is generated from a single microarray experiment has changed the focus of scientific experimental design and medical diagnoses. Because millions of genetic variations may be tested at one time by one microarray, the rational approach of generating “if-then” hypotheses derived from prior system knowledge have been obviated in favor of a data driven, hypothesis-free approach. Hence, unknown genetic samples may be analyzed for a host of human diseases, syndromes, and phenotypic states.

One type of genotyping microarray leverages the frequent and naturally occurring single nucleotide polymorphisms (SNPs) to interrogate the genome. It is currently believed that the human genome contains 4-7 million SNPs, all of which may be used as genetic biological markers for association with human diseases and variations. In other words, variations in the 1° structure of the human genome may either cause or segregate with genetic mutations that cause disease. Moreover, microarrays deliver highly quantitative information. Each genotype assayed by a microarray can be used to detect and measure genomic amplification and/or deletion events like aneuploidy and loss of heterozygosity. Current state-of-the-art microarray platforms interrogate millions of SNPs in a single array. At this density, a SNP is interrogated every 2-5 kb in the human genome, excluding centromeric and telomeric repetitive sequences.

Another genotyping microarray, comparative genomic hybridization (CGH), uses individual genomic loci spaced approximately 10 kb apart. While robust for some experimental applications, its lack of polymorphisms and probe tiling overlap preclude its use for many experimental designs.

Some microarrays exist for mitochondrial DNA but their sequence information and correlation to a specific mitochondrial disease is limited.

At the present time, no microarray platform has been successfully applied to single cell PGD genetic testing from human embryos for disorders exceeding a single or limited nucleotide sequence variation(s) (i.e. no improvement over the current single gene testing with the same limitations as previously described). To our knowledge, six IVF-PGD pregnancies have been reported using MDA and specific single gene PGD for cystic fibrosis, β-thalassemia, Marfan syndrome and Duchenne muscular dystrophy.

### SUMMARY OF THE INVENTION

Accordingly, it is an object of the present invention to provide a method for preparing genetic material from a single blastomere or trophectoderm cell(s) (optimized MDA) from an in vitro fertilized human embryo for the purpose of densely interrogating the content and primary structure of the genome.

It is another object to quantify and qualify specific nucleotide sequences from within the primary structure of the preimplantation human embryo genome for use as biological markers in a clinical diagnostic assay (preimplantation genetic diagnosis and screening).

It is another object to define the methods used to analyze the data arising from the genetic material from a single blastomere or trophectoderm cell(s) from an in vitro fertilized human embryo (Microarrays and fluorescence in situ hybridization (FISH)). The results of these analyses will identify all chromosome(s) aberrations, single gene disorders, genetic disorders segregating in families without a known DNA mutation and mitochondrial DNA mutations.

It is another object to improve grading of in vitro fertilized human embryos to assist the embryo selection decision making process for transfer. A more standardized and optimized embryo grading scheme is required to improve the embryologists' ability to choose the embryo with “the best” morphology that is most likely to achieve a successful pregnancy (as previously discussed in the background section).

It is still another object to quantify and qualify specific nucleotide sequences from within the primary structure of the human genome (nuclear) and mitochondrial DNA for use as biological markers in a clinical diagnostic assay (prenatal, neonatal, pediatric and adult genetic testing and screening). This includes genetic testing for all chromosome abnormalities, testing for transplant success (i.e. sex of organ in other sex host or “fingerprinting” to confirm a successful transplantation), to determine genomic (nuclear) or mtDNA sequence alterations in patients with a possible genetic disorder and to perform a global sub-telomeric microarray scan to identify cryptic aberrations associated with mental retardation and/or developmental delay.

One object is to perform all pre in vitro fertilization genetic and infectious disease screening of adult partners preparing for IVF.

Another object is to perform non-invasive diagnostic testing on isolated fetal cells circulating in maternal blood. This will eliminate miscarriage risks due to prenatal testing such as choronic villi sampling or amniocentesis.

It is still another object to optimize in vitro growth and development of embryos by identifying different proteins required by the embryo for optimal in vitro culture conditions.

Another object is to identify gene expression patterns in developing embryos.

Still another object is to identify pathways involved in infertility. This will be achieved by determining gene expression patterns in developing embryos, oocytes, sperm, endometrial biopsies and blood from both male and female partners. These gene expression patterns will be correlated with outcomes.

Another object is to determine a high-resolution amplification and/or deletion map to identify nucleotide sequences that may be associated with cancer and/or other genetic conditions.

It is a further object to determine which parent contributes the extra chromosome in PGD aneuploidy screening and which embryo implanted (by a modified DNA fingerprinting and genotype/haplotype markers).

It is another object to optimize strategies to successfully implement elective single embryo transfer (eSET). In conjunction with the previously described object discussed above on an improved embryo grading system, eSET will reduce the significant risk to couples for multiple births by reducing the numbers of genetically normal and embryogically graded “best embryo(s)” transferred.

It is another object to determine epigenetic aberrations due to methylation, hypoacetylation changes and/or other changes in the basal state of the physical structure of the genome (i.e. RNAi's, etc).

It is another object to determine disease predisposition using polymorphic and/or single nucleotide sequence variation and/or genotype/haplotype patterns.

According to the present invention, the above-described and other objects are accomplished by providing a method for genetic testing of human embryos for chromosome abnormalities, segregating genetic disorders with or without a known mutation, and mitochondrial disorders following in vitro fertilization (IVF), embryo culture and embryo biopsy, comprising the following steps:
1. Optimized embryo grading system
2. Less-invasive embryo biopsy technique with reduced cellular contamination
3. Optimized DNA extraction and amplification protocol for single cell analyses
4. Perform preimplantation genetic diagnosis and screening for aneuploidy and structural chromosome abnormalities using microarrays
5. Use fluorescence in situ Hybridization (FISH) to identify some types of chromosome aberrations.
6. Perform a modified DNA fingerprinting protocol
7. Determine imprinting and epigenetic changes in developing embryos
8. Perform genome-wide scans to identify genotype/haplotype patterns so as to clarify/diagnose possible multi-factorial genetic disease (i.e. bipolor disorder, etc) and to determine genotype/haplotype patterns that may predict future disease.
9. Determine single gene disorders with or without a known DNA mutation.
10. Determine mtDNA mutations and/or the combination of mtDNA and/or genomic (nuclear) DNA aberrations that cause genetic disease.

All of the above described genetic testing is the result of an improved and modified multiple displacement DNA amplification protocol and interrogating the content and primary structure of genomic (nuclear) and/or mtDNA through a modified and improved microarray platform. These analyses can be done on day-3 cleaving embryos, frozen/thawed cleaving embryos and fresh or frozen/thawed blastocysts.

### BRIEF DESCRIPTION OF THE DRAWINGS

Other objects, features, and advantages of the present invention should become more apparent from the following detailed description of the preferred embodiment and certain modifications thereof when taken together with the accompanying drawings in which:

FIG. 1 is a flow diagram of the method for in vitro fertilization (IVF) and genetic testing of human embryos according to the present invention.

FIG. 2 illustrates removal of a single blastomere for genetic analysis from a 9 cell day 3 embryo.

FIG. 3 illustrates the polymerase chain reaction.

FIG. 4 illustrates DNA amplification according to our MDA experimental protocol

FIG. 5 illustrates the Target DNA preparation for Microarray Analysis.

### DETAILED DESCRIPTION OF THE PREFERRED EMBODIMENTS

The present invention is a method to optimize embryo grading, to provide a less invasive and contamination-free embryo biopsy protocol, to perform a modified and enhanced multiple displacement DNA amplification protocol and a modified microarray platform for preimplantation genetic analysis.

The novel concept of the present invention is the ability to reduce the subjectivity associated with embryo grading, to reduce potential damage to biopsied embryos and to eliminate exogenous cellular contamination, to amplify adequate amounts of DNA from single cells without exogenous DNA contamination and to perform genetic testing on single cells from embryos prior to transfer within an IVF setting.

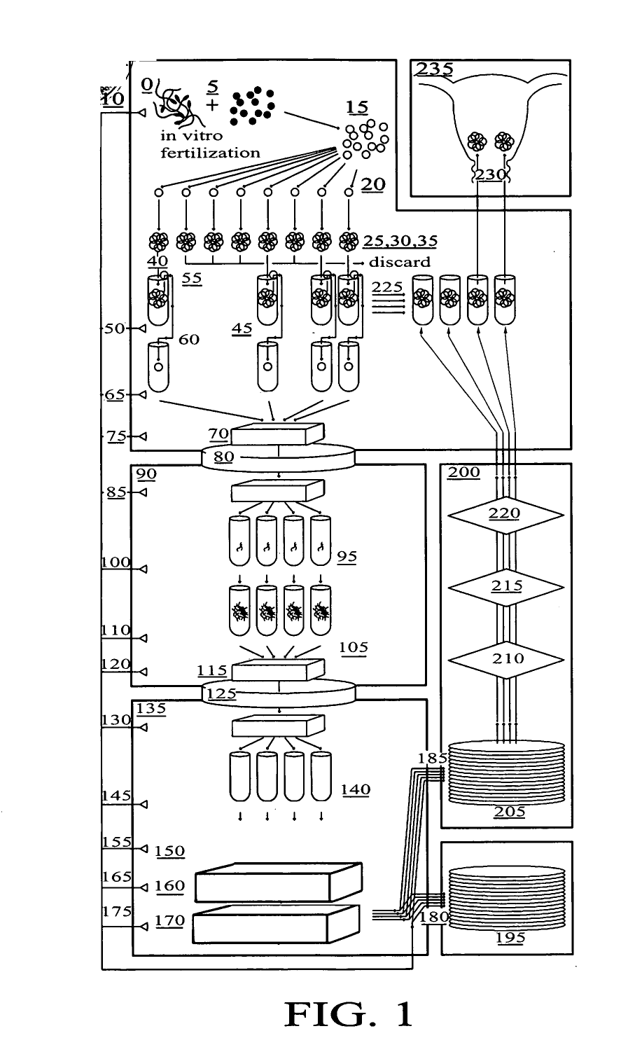

FIG. 1 is a flow diagram of the method for in vitro fertilization (IVF) and genetic testing of human embryos according to the present invention, in which the following components are referenced as shown:
0 Positive Pressure Embryology Laboratory
5 In Vitro Fertilization
10 In Vitro Fertilization Quality Control Checkpoint
15 In Vitro Embryo Culture
20 Day 1 Zygote Culture
25 Day 3 Embryo Culture
30 Visual Inspection Of Embryo Culture
35 Embryo Quality Scoring And Selection
40 Embryo Transferred To An Individual Culture Dish
45 Individual Embryos Assigned Unique Identification Number
50 Embryo Selection Quality Control Checkpoint
55 Single Cell(s) (Blastomere or Trophectoderm) Embryo Biopsy (If a trophectoderm biopsy is performed the biopsy and subsequent steps will begin once the embryo reaches the blastocyst stage)
60 Single Cell Transferred To Individual Assay Tubes. If FISH is also required, the single cell will first be fixed onto a glass slide as described below for FISH analysis and then the described DNA extraction protocol and microary analysis will be employed.
65 Transferred Cell(s) Quality Control Checkpoint
70 Opaque Transport Rack
75 Embryology Side Transport Quality Control Checkpoint
80 Ultraviolet Light Decontamination Transport Portal
85 Genome Amplification Side Transport Quality Control Checkpoint
90 Positive Pressure Genome Amplification Laboratory
95 DNA Extraction
100 DNA Extraction Quality Control Checkpoint
105 Multiple Displacement Amplification
110 DNA Amplification Quality Control Checkpoint
115 Opaque Transport Rack
120 Genome Amplification Side Transport Quality Control Checkpoint
125 Ultraviolet Light Decontamination Transport Portal
130 Molecular Biology Side Transport Quality Control Checkpoint
135 Normal Pressure Molecular Biology Laboratory
140 Target Sample Preparation
145 Target Sample Quality Control Checkpoint
150 Target Sample Arraying
155 Target Sample Arraying Quality Control Checkpoint
160 Target Sample Imaging
165 Target Sample Imaging Quality Control Checkpoint
170 Genotype And Copy Number Acquisition
175 Genotype And Copy Number Acquisition Quality Control Checkpoint
180 Data Transport To Off-Site Secure Server Farm
185 Data Transport To Genetics Analysis Laboratory
190 Off-Site Secure Server Farm
195 Secure Off-Site Server
200 Genetics Analysis Laboratory
205 Genetics Analysis Laboratory Secure Server
210 Quality Control Analysis Of Data And Process
215 Data Analysis
220 Testing Results And Embryo Selection
225 Embryo Retrieval From Culture
230 Embryo Transfer
235 Medical Clinic

The process begins in a POSITIVE PRESSURE EMBRYOLOGY LABORATORY 0. This laboratory is a clean-room environment with positive atmospheric pressure relative to all other rooms in the facility. In addition to IN VITRO FERTILIZATION 5, this laboratory functions as a clean area and all surfaces and ventilation are designed to control DNA contamination. At multiple key points throughout the entire process, various types of data are collected and used to insure the quality of the process and of the resultant genomic data.

An IN VITRO FERTILIZATION QUALITY CONTROL CHECKPOINT 10 is an initial quality control checkpoint at which the conditions of the in vitro fertilization event are defined and sperm and eggs are associated to their respective donors using a medical records system.

Fertilized eggs are then cultured in an IN VITRO EMBRYO CULTURE 15. The process of embryonic development is periodically monitored from the initial moment of fertilization through DAY 1 ZYGOTE CULTURE 20.

During DAY 3 EMBRYO CULTURE 25 a VISUAL INSPECTION OF EMBRYO CULTURE 30 is performed via light microscopy. At this time embryonic development is prescreened in accordance with an initial EMBRYO QUALITY SCORING AND SELECTION 35. Embryos that meet gross morphological criteria are moved forward in the analytical selection process and individual EMBRYOS are TRANSFERRED TO INDIVIDUAL CULTURE TUBES 40 (depending upon the newest and best culture conditions, the laboratory may employ group embryo culture for optimal in vitro conditions). Upon transfer to individual tubes, INDIVIDUAL EMBRYOS are ASSIGNED UNIQUE IDENTIFICATION NUMBERS 45. Before proceeding in the process, embryos must pass an EMBRYO SELECTION QUALITY CONTROL CHECKPOINT 50 to record relevant assay and sample metrics.

Next, SINGLE CELL (BLASTOMERE OR TROPHECTODERM(S)) EMBRYO BIOPSY 55 is performed and the CELLS are TRANSFERRED TO INDIVIDUAL ASSAY TUBES 60. All embryos are returned and maintained in culture 40. Individual biopsied cell(s) are recorded at a TRANSFERRED BLASTOMERE QUALITY CONTROL CHECKPOINT 65.

Transferred cells in individual culture tubes are placed in an ultraviolet light impervious OPAQUE TRANSPORT RACK 70. An EMBRYOLOGY SIDE TRANSPORT QUALITY CONTROL CHECKPOINT 75 is taken to record which embryos are placed in which rack and when that event occurs. Samples within the opaque transport racks are moved between rooms through an ULTRAVIOLET LIGHT DECONTAMINATION TRANSPORT PORTAL 80 that bombards the external surfaces of the transport rack with UV light, cross linking any contaminating DNA on the rack surface and maintaining the integrity of the POSITIVE PRESSURE GENOME AMPLIFICATION LABORATORY 90. A GENOME AMPLIFICATION SIDE TRANSPORT QUALITY CONTROL CHECKPOINT 85 is recorded.

DNA EXTRACTION 95 is then performed individually on all the cells and DNA EXTRACTION QUALITY CONTROL CHECKPOINT 100 is recorded.

A key step in the inventive process is a modified Multiple Displacement Amplification Protocol 105 and it for this reason that strict “clean-room” hygiene must be maintained between and within both positive pressure laboratories 0 & 90. The quality and quantity of the amplified genomic DNA is recorded at a GENOME AMPLIFICATION QUALITY CONTROL CHECKPOINT 110.

Following multiple displacement amplification of DNA, all samples are placed into an OPAQUE TRANSPORT RACK 115 and their disposition is recorded at a GENOME AMPLIFICATION SIDE QUALITY CONTROL CHECKPOINT 120. Samples within the opaque transport racks are moved between rooms through a unique ULTRAVIOLET LIGHT DECONTAMINATION TRANSPORT PORTAL 125 that bombards the external surfaces of the transport rack with UV light, cross-linking any contaminating DNA on the rack surface and maintaining the integrity of the POSITIVE PRESSURE GENOME AMPLIFICATION LABORATORY 90. A MOLECULAR BIOLOGY SIDE QUALITY CONTROL CHECKPOINT 130 is recorded when samples enter the NORMAL PRESSURE MOLECULAR BIOLOGY LABORATORY 135.

Molecular targets are synthesized from samples of amplified DNA of the biopsied cells during TARGET SAMPLE PREPARATION 140. A TARGET SAMPLE PREPARATION CHECKPOINT IS RECORDED 145 prior to TARGET SAMPLE ARRAYING 150. During this step, prepared targets are hybridized to the surface of relevant genotyping microarrays, and a TARGET SAMPLE ARRAYING QUALITY CONTROL CHECKPOINT 155 is marked. There are a large number of standardized and customized high-density genotyping array platforms that may be interchangeably employed for this purpose. These platforms may be provided by the following companies or other unspecified companies: Illumina, Affymetrix, ParAlelle, Nimblegen, Nanogen, Agilent, Perlegen, Sequenom. The hybridized samples are scanned at TARGET SAMPLE IMAGING 160 on equipment correspondent to the array platform and a TARGET SAMPLE IMAGING QUALITY CONTROL CHECKPOINT 165 is recorded. From the resulting scanned images, GENOTYPE AND COPY NUMBER ACQUISITION 170 is performed and a GENOTYPE AND COPY NUMBER ACQUISITION QUALITY CONTROL CHECKPOINT 175 records the event.

All data for each sample at all quality control checkpoints are associated with the relevant genotype dataset and the DATA IS TRANSPORTED TO A SECURE OFF SITE SERVER FARM 180, 190. The data is archived on a SECURE ON OR OFF SITE SERVER 195 for backup and disaster management.

Likewise, all data for each sample at all quality control checkpoints are associated with the relevant genotype dataset and the data is transported to GENETICS ANALYSIS SECURE SERVER 185 205. The GENETICS ANALYSIS LABORATORY 200 is essentially a dry laboratory that practices strict information hygiene. Before any of the genotypes or copy number information is analyzed, a QUALITY CONTROL ANALYSIS OF DATA AND PROCESSES 210 is performed to assure that the data is correctly assigned to the originating embryo and that all phases of the assay occurred under standard conditions. Passing this, true DATA ANALYSIS 215 is performed and then TESTING RESULTS AND EMBRYO SELECTION 220 is executed. Pending these results, EMBRYO(s) are RETRIEVED FROM CULTURE 225. The selected embryos are moved to the MEDICAL CLINIC 235 and EMBRYO TRANSFER 230 is completed.

A more exhaustive description of the above-described methods for embryo grading, embryo biopsy, DNA amplification and interrogating the content and primary structure of genomic (nuclear) and/or mtDNA through a modified and improved microarray platform for single cell genetic analysis, is herein described.

Embryo Grading—Cleavage And Development (Days 2-7 Post Oocyte Fertilization) Day 2 (Goal: 2-4 Cells)

Within the embryology laboratory of the IVF clinic, embryologists will remove the embryo culture dish(s) from the incubator(s) and check for the embryo cleavage status under high power microscopic magnification. Embryos should be 2 to 4 cells by 40 hr post-sperm fertilization. The embryos will then be sorted and grouped into separate wells according to cell stage. Note whether the patient should be transferred D3 or D5.

The appearance of each embryo, tech ID and the date of observation will be recorded on the worksheet. The dishes will be returned to the incubator(s) immediately. Embryo cell numbers should approximately double every 18 to 24 hours. Embryo quality should be evaluated according to Veeck, Atlas Of The Human Oocyte And Early Conceptus, Baltimore, Williams and Wilkins (1991).

As previously discussed in the background section, embryo grading is subjective with observational variations seen between embryologist observers. Therefore, one must significantly improve embryo grading as described in subsequent sections.

Day 3 (Goal: 6-8 Cells)

Oocytes not fertilized and oocytes that fertilized abnormally should be disposed as hazardous waste in the appropriately labeled receptacles. Disposal should be recorded on the patient's egg sheet. Embryo stage and quality should be recorded on the medical record and on the fertilization report.

All blastomere biopsied PGD embryos are done on day 3.

These embryos will be destined for culture to day 5, 6 or 7 prior to transfer.

All blastocyst biopsies of trophectoderm cells, or eventually cells obtained from the inner cell mass, will be done once the embryo reaches the blastocyst stage of in vitro embryo development. Blastocyst biopsies can be done on thawed or fresh embryos with a goal of a patient transfer 24 hours post biopsy.

Once day-3 embryos are biopsied, these should be moved after evaluation to a pre-equilibrated dish of blastocyst culture medium. The number of embryos cultured to day 5/6/7 is recorded on the worksheet.

Day 4 (Goal: Morula Stage)

Embryo stage should be recorded with tech ID, date and time of observation on oocyte sheet for those patients being cultured to day 5/6/7. Changeover into fresh blastocyst media will occur on this day. Embryos of patients who have received a day 3 transfer are not observed on day 4.

Day 5 (Goal: Blastocyst Stage)

Embryos should be graded based on their degree of expansion, inner cell mass (ICM) and trophectoderm (TE). As discussed in the background section, different IVF laboratories use different grading scales and observational variations occur between embryologists and laboratories. Clearly, an enhanced embryo grading scale is required for optimal in vitro embryo development.

Embryos graded and appropriate one(s) selected for transfer should be isolated into their own well. Embryos not transferred or cryopreserved on day 5 should be cultured two additional days.

Day 6/7 (Goal: Blastocyst Stage)

Embryos should be evaluated and graded then appropriate ones cryopreserved per patient consent.

Embryo Biopsy on Day-3 Cleaving Embryos

Make a 30 ul drop of media in a petri dish using a polished sterile pipette with Ca+/Mg free medium, 10% human serum albumin and overlay with mineral oil.

Make one dish for each embryo to be biopsied and label with the initials of the patient and the individual embryo number. Make 1-12 dishes at a time.

Move individually numbered embryos in to correspondingly numbered dishes.

Move the dish to the inverted microscope.

Identify the blastomere for biopsy. Make sure the blastomere has a visible nucleus but try to avoid removing the largest blastomere. Trophectoderm cells can also be biopsied from blastocysts*. If a trophectoderm biopsy is performed, incorporate Tween-20 and a smaller bore pipet to assist in the separation of individual cells. All other described protocols are the same except when one removes trophectoderm cells.

Position the embryo so the targeted blastomere is adjacent to the biopsy pipet.

Focus on the area of the Zona Pellucida (ZP) to be removed. Switch to the laser objective.

Adjust the laser and use 200-400 ms bursts at 100% power to remove small pieces of the ZP. Continue to remove ZP until the hole is complete and the ZP is breached with ˜30 micron hole in the zona pellucida at the site most accessible to the blastomere. Note: The top edge of the hole should be perpendicular to edge of the ZP while the bottom edge of the ZP should tapered to a 45 degree angle to the outside of the embryo.

Using a 10-40 um pipette, aspirate the desired cell into the pipette. Using slow light suction and moving the pipette over the blastomeres will minimize cell rupture. Withdraw the blastomere slowly to the outside of the embryo.

Place the embryo back into its own dish that is pre-labeled with it the patients name, unique patient identification number and the embryo(s) number. Use several rinse drops in the perimeter of the dish before putting the embryo into the center drop.

The dish containing the blastomere biopsied embryo should be returned to the incubator for blastocyst culture and leave the blastomere in the biopsy dish until cell fixation.

Repeat steps for all day-3 embryos to be biopsied. Make sure to label all of the biopsy dishes before starting.

*Trophectoderm Biopsy—One significant risk of a day-3 embryo biopsy is damaging the embryo during the procedure with a subsequent risk to embryo development in vitro, a potential reduction in implantation and a possible increase in the prevalence of biochemical pregnancies.

To reduce the potential damage to the biopsied embryo with the subsequent increase in the live birth of healthy babies, an improved embryo biopsy technique and PGD process is required. Recently, in collaboration with others, we've optimized blastocyst-trophectoderm biopsy and PGD analyses on fresh or frozen blastocysts. This biopsy and PGD technique employs the removal of trophectoderm cells (which eventually develop into the placenta) and the completion of PGD within 24-hours of embryo biopsy. This enhanced biopsy technique may reduce the risk of damaging the embryo during biopsy, reduce the risk of a lower implantation rate and permit a more accurate PGD analyses to be performed.

The above described biopsy protocols on day-3 blastomeres and/or trophectoderm cells employs a laser. Biopsy methods employing acid tyrodes, a mechanical methodology or other unforeseen techniques should also be considered in the development of a less invasive biopsy protocol so as to reduce embryo damage.

For microarray analysis, the single cell goes into a pre-labeled tube. For FISH, the cell is lysed and fixed on a glass microscope slide (see hypotonic swelling and cell fixation below).

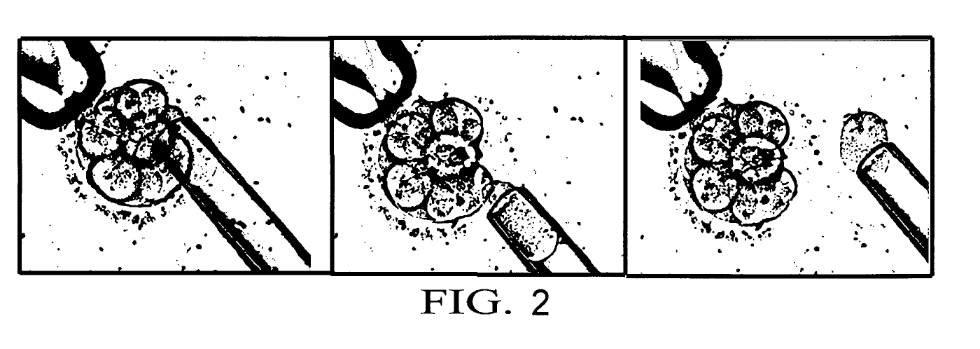

FIG. 2 illustrates removal of a single blastomere for genetic analysis from a 9 cell day 3 embryo.

Validation Steps to Insure No Cellular Contamination Following Embryo Biopsy

To optimize embryo biopsy and to reduce cellular contamination prior to Genetic testing for microarray analyses or other DNA nucleotide or structural experimental protocols all IVF/embryo biopsy personnel must do the following validation steps.

The cells analyzed for contamination can be blastomeres from day-3 embryos or trophectoderm cell(s) (and possibly inner cell mass cells) from in vitro cultured blastocysts. The isolated cell is transferred to a 0.2 ml thin-walled PCR reaction tube containing 5 μl of cell lysis buffer.

The Genetics laboratory will supply empty 0.2 ml thin-walled PCR reaction tubes and a bulk tube of working cell lysis buffer (this is usually 0.2M KOH). The IVF lab will add 5 μl of the working cell lysis buffer to the individual tubes as biopsies are performed and add the single cell to each appropriately labeled tubes.

Establish Contamination Rate

Each IVF lab should demonstrate competency by supplying 3 sets of ten single cells with two wash blanks per single cell.

The cells can be from fresh or frozen embryos.

The remainder of the embryo may/or may not be included. If submitted, the embryo should be washed under the same conditions as the single cells, however wash tubes for the embryo are not required to be submitted.

Establish Amplification Rate

The IVF lab should demonstrate competency by supplying 3 sets of ten single cells with two wash blanks per single cell.

The single cells must be from fresh embryos only.

The remainder of the embryo after biopsy must be included. The embryo should be washed under the same conditions as the single cells, however wash tubes for the embryo are not required to be submitted.

The data from the 6 sets of single cells should be examined for 1) the absence of contamination in wash blanks, 2) amplification of a single DNA fragment of the correct size from single cells, and 3) correlation between the DNA diagnosis of the single cell and the remainder of the embryo (embryo mosaicism must be considered)

IVF labs/biopsy groups should be rated as competent for embryo biopsy after the Genetics laboratory examines the three sets of ten blastomeres from fresh donated embryos and finds that:
wash blank contamination is ≦1%
PCR amplified DNA in ≧93% of single cells
   Fluorescence in situ Hebridization (FISH) Hypotonic Swelling and Cell Fixation Identification of Slides

Patient name and embryo number

Slide map should identify each biopsied nucleus on each slide.

Laboratory Preparation

Set humidity to 34-42%.

Prepare fresh fixative solution.

Prepare fresh hypotonic solution

Make a dish with two drops of hypotonic solution

Pull a glass 9″ glass pipette near the neck. Then pull it again to make a very fine dispenser pipette. Fit a pipette bulb on the other side. Place 4 ml of fixative in a 10 ml graduated glass cylinder and aspirate some fixative in the dispenser pipette. Leave the dispenser pipette in the graduated cylinder.

Make a cell transfer device.

Place the mirror of the stereoscope at a 45-60 degree angle by which the cell placed on a glass slide will look three-dimensional.

Procedure

Use the cell transfer device to move the cell from the biopsy dish to the dish with the two drops of hypotonic solution.

Transfer the cell from one drop of hypotonic to the other. The purpose of this is to clear the transfer needle of oil. Make sure before picking the cell from the second drop that there is no oil in the pipette of the transfer device.

Drop the cell onto the glass slide.

Immediately and without losing visualization of the cell, hold the glass slide with one hand and pick up the fixative dispenser with the other.

Approach the tip of the fixative dispenser to the cell so a blurry shadow of it can be seen under the stereoscope. That is approximately 1 cm above the cell.

Before the drop of hypotonic solution containing the cell dries, add a single drop of fixative to the cell.

The cell moves. Follow it under the stereoscope.

Once the cell stops, drop a second drop of fixative. Usually the cell does not move this second time because it is stuck to the slide.

The biopsied cell will lyse and disperse all cellular structures. The remaining nucleus will be the target for FISH analysis.

Before the fixative dries blow air over the nucleus. This air pushes most of the remaining cytoplasm away from the nucleus. Draw a small circle around the nucleus with the diamond pencil. The smaller the circle, the easier one can find the nucleus.

Look at the nuclei using a phase contrast microscope. Nucleus? Fragments? Cytoplasm removed? Cell lysed? If no intact nucleus is present from a given cell or the cell has failed to lyse repeat the biopsy and cell fixation.

Place as many nuclei as possible on a single slide.

Within our invention, we will primarily employ FISH when our optimal multiple displacement DNA amplification and modified microarray experimental technique is not possible due to target cell DNA sequence variations (such as highly repetitive sub-telomeric sequences, etc) or other unforeseen technical issues.

Prepare Target DNA on Slide for Hybridization

Place slide into a 1×-3× SSC solution, pH=˜7.2 for 2 minutes at room temp.

Dehydration of Slide

Incubate slide in ice cold 50% ethanol for 2 minutes.

Incubate slide in ice cold 75% ethanol for 2 minutes.

Incubate slide in ice cold 95% ethanol for 2 minutes.

Allow slides to drip dry.

Place slide on shelf in 37° C. incubator to warm, dry and equilibrate to optimal hybridization temperature.

Retrieve slide from incubator when ready to add probes.

Prepare Probe Mix

For numerical FISH analyses using alpha or beta satellite or other repetitive DNA probes

5-30 ng per probe in an approximate 10 ul hybridization mix so as to optimize the chemical kinetics of probe DNA to target DNA.

Hybridization Solution—This varies depending upon the type of DNA probe used (ie. Satellite DNA, locus specific probe, etc). Chemicals and repeat sequence blocking DNA requirements may include formamide, dextran sulfate, SSC, sequence specific blocking materials on target DNA and other currently unknown items for optimal DNA probe hybridization.

For FISH Analysis Using Locus Specific DNA Probes

5-30 ng per probe in an approximate 10 ul hybridization mix so as to optimize the chemical kinetics of probe DNA to target DNA. These probes can be run in combination with the above described satellite DNA probes.

For Structural Chromosome Aberrations Analyzing Telomere or Sub-Telomeric Sequences

5-40 ng per probe in an approximate 10 ul hybridization mix so as to optimize the chemical kinetics of probe DNA to target DNA.

Preparing & Applying Probe Mix

In an eppendorf tube, combine probes and hybridization solution as calculated.

Make sure that the probes and hybridization solution have come to room temperature, are centrifuged, vortexed, and centrifuged again.

Pipet 10 ul (total volume of mix from tube) onto hybridization site.

Place appropriate coverslip onto hybridization site(s).

Press coverslip lightly with forceps to remove air bubbles, and to distribute mix evenly within coverslipped area.

Create Moisture (Hybridization) Chamber

In a 1000 ml beaker, moisten folded paper towels and place at bottom of beaker.

Place a slide rack into beaker and use aluminum foil to cover top of beaker.

Place chamber into 37° C. incubator.

These hybridization chambers can be modified using other equipment (i.e. Abbott VYSIS HyBrite system).

Denature Target and Probe DNA Begin Hybridization

Place slide onto a 75-80° C. hot plate for 30 sec to 8 min depending upon the sample.

Rapidly transfer the slide to moisture or hybridization chamber.

Cover moisture or hybridization chamber and place back into 37° C. incubator.

Note both denature time and hybridization time.

Stringency Washes

Preheat 0.2-0.8×SSC to 35 to 77° C. in a wash chamber.

Place slides in the 0.2 to 0.8×SSC.

Allow the cover slip(s) to fall off.

Then, incubate the slide for 1 to 5 min @ 35-77° C.

Wash slides in the second stringency wash of 1-4×SSC, 0.1% NP-40 @ RT for 30 sec to 3 minutes.

Remove the slide from the solution and let drip dry. Do not allow the slide to dry completely.

An alternative stringency wash protocol can employ lower SSC concentrations along with higher temps or visa versa.

Visualize Slides

Take antifade counterstains out of the −20° C. freezer and allow it to thaw in a 37° C. water bath.

Centrifuge the counterstain tube at approximately 16,000 rpms

Vortex tube, and spin down.

Add approximately 10 uL of counterstain to each hybridization site on the slide.

Cover the droplet(s) with an appropriate coverslip. Use forceps to remove any bubbles.

Allow the slide to set in the dark for a few minutes, approximately 10 min.

Scoring

Use an epifluorescence microscope equipped with multiple excitation filters.

Record number of fluorescent signals for each chromosome.

Minimize between-scorer subjective variability by discussing uncertainties with only one other person.

Diploid is defined as one having two signals for each analyzed chromosome.

Document scoring results on appropriate FISH scoring sheets.

Destaining & Rehybridizing Probes

Place slide in 1×PBS for 20 minutes and shake on a rotator at 100-300 rpm.

Dehydrate slide in 50, 75, and 95% ethanol as in above described steps.

Add appropriate second round of DNA probes within optimal hybridization mix. Follow the above described experimental steps except increase the denaturation time due to the altered denaturation conditions required for a second (or third) round of analyses. Note scoring on FISH scoring sheets.

If a third round of hybridization is required, repeat the above except increase the denaturation times for reasons previously described.

Preimplantation Genetic Diagnosis for Aneuploidy and Structural Chromosome Abnormalities Using Microarrays

The largest hurdle in performing PGD on a single cell is getting enough DNA without introducing experimental artifact. Only approximately 6 picograms of genomic (nuclear) DNA exits within a single human blastomere or trophectoderm cell. In order to run a microarray analysis, one requires approximately 250 nanograms to successfully complete the assay. Therefore one must incorporate additional DNA amplifications to attain the required amount of genomic DNA. Polymerase chain reaction (PCR) can be employed using universal primers to attain the required amount of DNA. However, this methodology induces experimental artifacts that result in preferential regions of amplification and/or deletion and/or other sequence specific issues due to PCR.

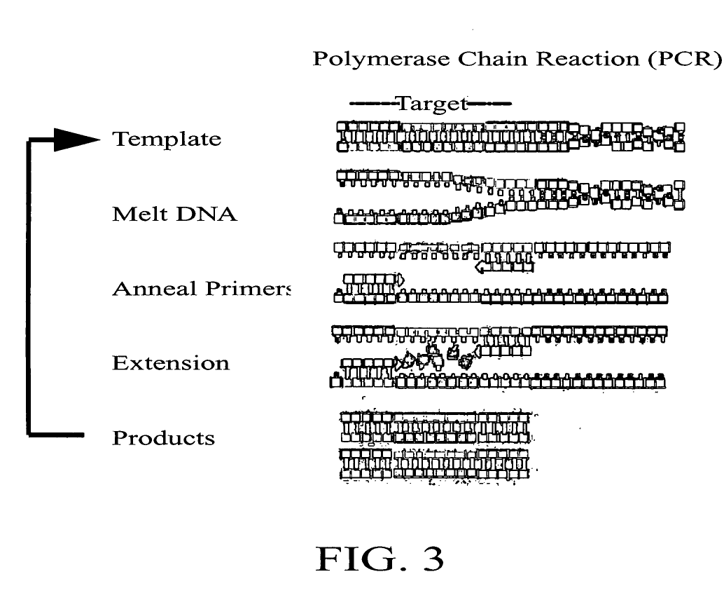

FIG. 3 illustrates the polymerase chain reaction. Because of this, a new methodology is required.

Therefore, we employed a modified version of Multiple Displacement Amplification (MDA) using bacteriophage Φ29 DNA polymerase- and exonuclease-resistant random hexamer primers to obtain linear amplified DNA with the limited and inconsequential introduction of experimental artifacts. Our modified multiple displacement amplification protocol has additional advantages over previously known DNA amplification methods. These include but are not limited too: 1) it's high processivity of Φ29 DNA polymerase that generates fragments of ˜≧10 kb, 2) the enzyme has better proofreading activity that results in lower mis-incorporation rates (˜1 in 10⁶-10⁷) compared to Taq DNA polymerase (˜3 in 10,000) and 3) the amplification of mtDNA.

Potential limitations of MDA include the following: The inability of commercial kits to produce adequate DNA for single cell genetic testing, the potential for non-specific DNA amplification most likely due to primer directed, template independent DNA synthesis, thus DNA yields measured by routine techniques may be inaccurate and allele drop-out at heterozygous loci. The MDA reactions also can be catalyzed by Bst or Φ29 DNA polymerase but Bst amplified DNA products produce lower yields with erroneous amplified DNA sequences. Contamination is also a significant issue with MDA of single cells because of Φ29 DNA polymerases fidelity and its ability to amplify all DNA sequences within the amplification reaction tube.

Hence, in addition to our modified and enhanced MDA protocol using Φ29 DNA polymerase for DNA extraction and amplification, we also employed a specialized DNA quantitation method called Pico Green. This fluorometric DNA quantification method more accurately quantifies the amount of DNA obtained from amplified DNA, minimizes the contributation of RNA or ssDNA and quantifies intact dsDNA amplified by our modified MDA protocol. We will also employ other modified experimental techniques, such as Taq-Man PCR and others, to ensure that we obtain adequate DNA coverage and heterozygous allele amplification for genetic analysis. Our protocol significantly reduces the risk of a random under- or over-amplification of DNA in single cells and optimizes the amplification protocol to obtain suitable DNA for single cell genetic testing.

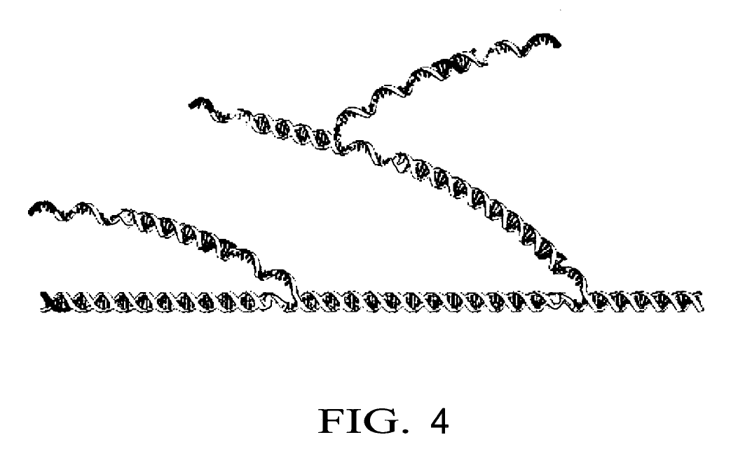

FIG. 4 illustrates the DNA amplification, that is not iterative annealing and denaturing, therefore the amplification is linear.

We optimized molecular genetic experimental techniques to successively isolate and amplify DNA from single cells and to run a modified microarray platform in order to achieve an acceptable detection rate (hybridization efficiency) and call rate (the success rate identifying the genotype of each hybridization) for the purpose of successfully employing microarray platforms for comprehensive genetic testing on single cells.

An enhanced and optimized embryo biopsy procedure using a laser (as previously described) was performed on day-3 cleaving embryos (n=5).

Single blastomeres were placed in individual, sterile, Eppendorf tubes containing 60 ul of Hanks+5 mM EDTA. We then performed an optimized multiple displacement DNA amplification protocol employing random hexamer primers and Φ29 phage polymerase. Specifically, a total reaction volume of 750 ul included 37 mM Tris-HCl, pH 7.2, 50 mM KCl, 10 mM MgCl₂, 5 mM (NH4)₂SO₄, 1 mM dNTPs, 50 uM exonnuclease-resistant hexamer, 1 unit/ml yeast pyrophosphatase and 800 units/ml of Φ29 DNA polymerase. The reactions were incubated for 18 hrs at 30° C. and stopped by heating to 65° C. for 3 mins. The DNA was digested with Xba I, ligated with universal primers and amplified to approximate 1000 kilobase (kb) size fragments. The DNA was randomly nicked with DNase I to produce fragments of approximately 500 base pairs (bp). We completed an adapter ligation step according to routine protocols. We then performed a one cycle PCR amplification step to reduce DNA complexity. The DNA was then fragmented and end labeled with fluorophores using routine methods. We then used invariant DNA genomic markers to determine whether the entire genome was amplified without experimental induced regions of amplification and/or deletion using target DNA markers on each chromosome arm. We performed TaqMan PCR to ensure heterozygous allele amplification for each chromosome marker. We used an Affymetrix 100 k DNA microarray and platform comprised of single nucleotide polymorphic markers (SNPS). We fluorescently labeled single blastomere DNA and used automated fluorescence image capture and software scoring for genomic analysis. Using these arrays, there is massive target DNA redundancy of approximately 80 different probes per SNP. We determined the fluorescent detection rate following each hybridization and success rate identifying the genotype of each hybridization.

Our initial amplification and analysis on five DNA samples yielded 597 ug+/−84 ug of DNA per single cell and showed genomic coverage >75% with heterozygous allele detection in 3/5 samples. Following microarray analysis our initial detection rate ranged from 63.9 to 78.1% with a genotype call rate 46.2 to 48%. We contributed these poor results to the overamplification of genomic DNA (>500 ug) and/or operator induced contamination. Our poor initial results could be due to the following:
Poor quality embryos. The embryos were 3 pns (genetically abnormal) and donated for research under an IRB approved study only permitting us to use genetically abnormal embryos.
Possible exogenous contamination during the biopsy and transfer of cells to the Eppendorf tubes.
Contamination during the DNA extraction.
Over amplification of DNA with subsequent variations in amplified DNA sequence due to experimental issues and/or contamination.
Final total volume of reaction mix? Too dilute?

We then used 2 pn aneuploid abnormal embryos and an optimized laser biopsy protocol for all additional analysis.

We then optimized our DNA extraction and amplification protocol to address all questions raised during the first experiment, to reduce the amount of amplified DNA and to reduce or eliminate exogenous DNA contamination. We added an initial double wash step in phosphate buffered saline (PBS) of each biopsied cell prior to cell transfer into each individual Eppendorf tube. We performed all cell isolations in a laminar-flow hood and all DNA extractions, linear amplifications, digests and labeling in a “PCR Workstation”. The laminar-flow hood reduces exogenous contamination from the person performing the biopsy and/or the surrounding environment. The “PCR Workstation” protects against DNA contamination, by using UV irradiation to block replication of contaminating DNA sequences by causing adjacent pyrimidines to undergo dimerization. It also protects against cross or airborne contamination by limiting exposure of lab workspace to the remainder of the laboratory. The next 5 samples produced a lower concentration of amplified DNA from each single cell (50 ug+/−7 ug) with a genomic coverage >85% and heterozygous allele detection in 4/5 samples. Our microarray analysis detection rate ranged from 84.1% to 88% with a genotype call rate 79 to 88%. We concluded that we successfully lowered the amount of amplified DNA and reduced our exogenous DNA contamination but still needed to increase our heterozygous allele detection rate, our hybridization efficiency rate between probe and target DNA and our genotype call rate.

We therefore, further optimized our protocol using part of our previously described “invention” protocol. We also modified our DNA amplification protocol. Double PBS washed single blastomeres were placed in individual, sterile, Eppendorf tubes containing 5-60 ul of Hanks+5 mM EDTA. We then performed an optimized multiple displacement DNA amplification protocol employing random hexamer primers and Φ29 phage polymerase. Specifically, a total reaction volume of 40-800 ul included 37-42 mM Tris-HCl, pH 7.2, 50-60 mM KCl, 10-17 mM MgCl₂, 5-7 mM (NH₄)₂SO₄, 1-2 mM dNTPs, 50-75 uM exonnuclease-resistant hexamer, 1-2 unit/ml yeast pyrophosphatase and 800-1000 units/ml of Φ29 DNA polymerase. The reactions were incubated for 4-20 hrs at 30-32° C. and stopped by heating to 65° C. for 3 mins. These 10 samples produced a lower concentration of amplified DNA from each single cell (800 ng+/−27 ng) with a genomic coverage >98% and a heterozygous allele detection rate in 9/10 samples. The microarray analysis detection rate ranged from 95% to 98.1% with a genotype call rate ranging from 95 to 96%.

We further optimized our DNA extraction and multiple displacement amplification protocol from single cells. These modifications include but are not limited too: 1) Using PBS without calcium or magnesium, 2) Use nuclease-free bovine serum albumin, 3) Depending upon the manufacture of the Φ29 polymerase, one must store the enzyme either at −70° C. or −20° C., 4) One can incorporate a PCR DNA purification kit to purify DNA prior to microarray analysis as long as it maintains a high DNA yield, 5) Use autoclaved potassium hydroxide (KOH) in the denaturation step, 6) Only use albumin after 4° C. short-term storage (<˜1 week), 7) Optimize the concentrations of DTT and KOH within the alkaline buffer denaturation step so as to not damage the target DNA for amplification, 8) All media, etc used during embryo culture and embryo biopsy must be appropriately autoclaved and/or biopsied, 9) Avoid exogenous DNA contamination by using our described specialized lab conditions, 10) The use of neutralization buffer following cell lysis should be evaluated and included or excluded based upon final microanalysis evaluation, 11) Once the single cell is biopsied, add the cell to 5-60 ul of 0.5 M Hanks+5 mM EDTA, KOH or albumin, 12) During the amplification step, use a total volume between 40-750 ul and a reaction incubation time between 2 and 20 hours, 13) Incubate the amplification reaction at 29-33° C., 14) Use an alkaline DNA denaturation step and not a high temp (˜95° C.) step to avoid DNA damage, 15) Commercially available kits such as GenomiPhi v2 and Repli-g-Kit can be used for cell lysis, DNA denaturation and DNA amplification steps if exhaustive steps are included to reduce exogenous DNA contamination, improve DNA yield, improve genomic coverage and optimize heterozygous allele identification. Several microarray platforms have been evaluated and we determined that the Illumnia chip currently works best for our analyses.

Additional studies using the Illumnia CNV370 chip on >100 single cells isolated from cytogenetically abnormal and normal cell lines and blastomeres from human embryos showed in many cases, a genomic coverage >98%, a heterozygous allele detection rate >90% and a microarray detection rate and genotype call rate >90%. In some cases, these call rates exceeded 99%. These analyses documents our ability to provide a complete molecular karyotype for all 23-pairs of chromosomes, to determine genetic imbalances due to de novo or parental segregating reciprocal or Robertsonian translocations, to identify chromosome aberrations due to pericentric or paracentric inversions, to determine genetic disorders due to DNA duplications (i.e. Charcot-Marie-Tooth, type 1A) or microdeletion syndromes (i.e. DiGeorge syndrome).

Our current methodology for DNA extraction and amplification may at times be sub-optimal for amplifying sub-telomeric and/or highly repetitive DNA sequences. Therefore, for subtelomeric structural rearrangements, we may be required to first employ FISH to identify structural chromosome abnormalities (as previously described) prior to extracting the DNA from the fixed cell (according to routine laboratory protocols) and running an aliquot of DNA on our microarray platform.

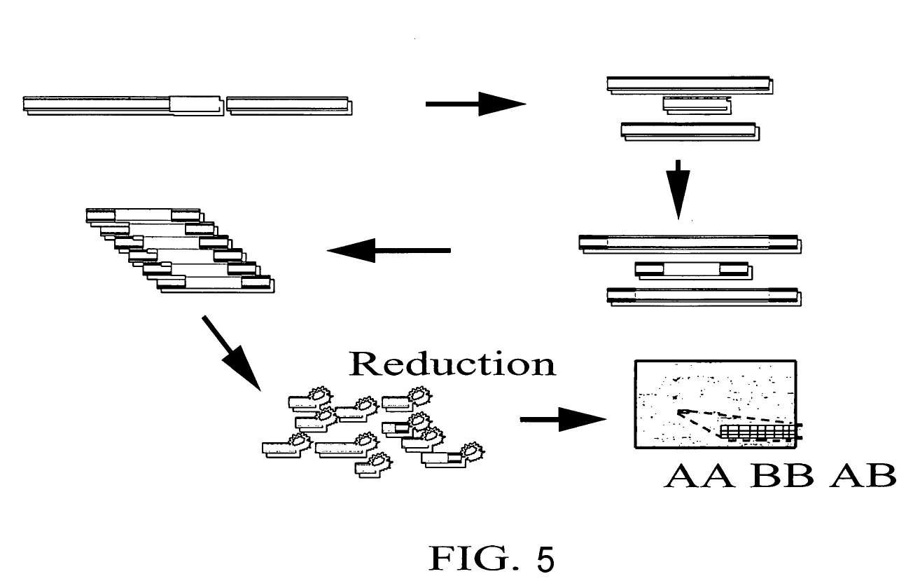

FIG. 5 illustrates the Target DNA preparation for Microarray Analysis.

Using our described invention, we will also provide the following genomic (nuclear) and mtDNA screening and diagnostics from a single cell or cells:

1. DNA fingerprinting—Using a modified microsatellite analysis and/or genotype/haplotype results from our microarray protocols, we will determine the “fingerprint” of all embryos, which embryo implanted, which partner provided the extra chromosome in PGD aneuploid diagnostics and which embryo to select for elective single embryo transfer (eSET).
Short tandem repeats (STRs) and/or microsatellite analysis will be performed on amplified DNA. The microsatellite markers used will be selected based on high heterozygosity between partners and within the normal population.
Genotype/haplotype information will be obtained from SNP, comparative genomic hybridization (CGH) or other above described array based DNA analyses.
mtDNA genotyping can clarify maternal transmission and Y-chromosome markers can clarify paternal transmission. These DNA typings can be used in paternity studies.

Using our described modified DNA fingerprinting technique along with genotype/haplotype DNA array information, we will optimize embryo grading to reduce embryologist subjectivity, observer variability and variations between IVF laboratory grading systems. Transferred embryos will be noted on the transfer sheet. Once a successful pregnancy is achieved, one will “fingerprint” fetal and/or baby DNA and correlate it with the “fingerprint” of the transferred embryos. Additional studies and statistical calculations will identify the best “fingerprint” to achieve a viable pregnancy and the delivery of a healthy baby.

We will determine what partner provided the extra chromosome (husband or wife) in an aneuploid embryo. One critical question for couples with aneuploid embryos is “who” provided the extra chromosome(s). This will provide important information to the couple considering all possible IVF or other options (donor sperm, donor egg, adoption, etc).

We will also select “the best” embryo for elective single embryo transfer (eSET). As described above, an optimized embryo grading system will provide a standardized protocol so as to select “the best” and most likely embryo to transfer to achieve a viable pregnancy. One significant complication of IVF is multiple births. Multiple gestations put the mother at risk for serious complications during pregnancy and multiple births generally require specialized and extended hospitalization. The incorporation of eSET into IVF clinics worldwide will reduce multiple birth complications. Additionally, some countries have laws limiting the number of embryos transferred based upon the age of the woman (i.e. only one embryo can be transferred for women ≦35).

2. We will determine the imprint status of each transferred embryo (ie uniparental disomy for Prader-Willi and Angelman syndrome, etc) so as to determine if both copies of a chromosome came from the same partner (husband or wife) or if epigenetic changes occurred during in vitro embryo development. We will achieve the first by our fingerprinting protocol described above. Epigenetic changes will be identified by using our amplified DNA as described and quantitative methylation analyses protocols based upon bisulfate DNA sequencing and/or methylation-specific multiplex ligation-dependent probe amplification. We may also employ other methods evaluating RNAi's or acetyl groups.

3. We will clarify/diagnose genetic disease (i.e. bipolar disorder) and determine disease predisposition using polymorphic and/or single nucleotide sequence variation and/or genotype/haplotype patterns. This will be achieved by identifying single nucleotide polymorphisms (SNPs), copy number variations (CNVs), target gene(s), taq SNPs (based upon HapMap populations), block substitutions, heterozygous or homozygous insertion/deletion events (indels), inversions, segmental duplications and/or other currently unknown genomic (nuclear) or mitochondrial DNA variations identified by our modified microarray analyses.

4. We will use our described DNA amplification and microarray analysis to determine single gene disorders with or without a known DNA mutation. For genetic disorders with a known genomic (nuclear) DNA mutation, we identify DNA changes on specifically prepared arrays.

The segregation of genetic disorders without a known DNA mutation can also be diagnosed using our described invention. This is accomplished when multiple family members are affected and one can do traditional genetic linkage analysis.

5. We will also use our described DNA amplification and microarray analysis to determine mtDNA mutations and/or the combination of mtDNA and genomic (nuclear) DNA aberrations that cause genetic disease.

Upon completion of the above described protocols, we can: Provide a comprehensive genetic screening and diagnostics on single cells from embryos prior to the transfer of genetically normal embryos within an IVF setting. We will determine a complete molecular karyotype for all 23-pairs of chromosomes, identify genetic imbalances due to de novo or parental reciprocal or Robertsonian translocations, pericentric and/or paracentric inversions, we will also identify duplication syndromes (i.e. Charcot-Marie-Tooth, type 1A), diagnose microdeletion syndromes (i.e. DiGeorge syndrome) and identify cryptic sub-telomeric chromosome rearrangements. Using a modified DNA fingerprinting technique and genotype/haplotype information, we will optimize embryo grading by identifying what embryo implanted by determining the best embryonic “fingerprint” most likely to achieve a viable pregnancy. The identification of the “most likely” embryo to implant and to achieve a viable pregnancy will also provide an optimized grading scheme for eSET. The incorporation of eSET into IVF clinics will reduce IVF associated multiple birth complications. This optimized embryo grading scheme could also assist IVF programs within countries that by law, limit the number of embryos transferred based upon maternal age. We will also determine what partner provided the extra chromosome in aneuploid embryos by our fingerprinting experimental protocol. We will identify the imprint status of embryos by our described fingerprinting protocol and identify epigenetic changes during in vitro embryonic development by identifying gene active and inactive sequences. We will clarify/diagnose genetic disease(s) and determine disease predisposition using nucleotide sequence variation(s) and/or genotype/haplotype information. We will also identify single gene disorders, with or without a known DNA mutation, segregating in families. We will identify mtDNA mutations and/or the combination of mtDNA aberrations with genomic (nuclear) DNA mutations that cause genetic disease.

It should now be apparent in light of the foregoing description that the present invention accomplishes the following: We will provide couples undergoing IVF an optimized and enhanced genetic screening technology that will significantly improve the couples dream of having a healthy, normal baby.

We claim:

1. A method for interrogating the content and primary structure of genomic (nuclear) and mtDNA in human embryos for comprehensive genetic screening and diagnosis using a microarray, comprising the steps of:
embryo grading;
embryo biopsy;
DNA amplification;
performing preimplantation genetic diagnosis and screening using a microarray platform for single cell genetic analysis.

2. The method for interrogating content and primary structure of genomic (nuclear) and mtDNA in human embryos using a microarray according to claim 1, further comprising an IVF step before said grading step, said IVF step including fertilizing human ova in vitro with human sperm under sterile laboratory conditions, and growing said fertilized human ova (zygotes) in an embryo culture.

3. The method for interrogating content and primary structure of genomic (nuclear) and mtDNA in human embryos using a microarray according to claim 1, wherein said step of embryo grading comprise assessing zygotes with a microscope and grading based on pre-defined morphological criteria.

4. The method for interrogating content and primary structure of genomic (nuclear) and mtDNA in human embryos using a microarray according to claim 1, wherein said step of embryo biopsy comprises transferring a single blastomere or trophectoderm cell(s) from a viable embryo for genetic analysis.

5. The method for interrogating content and primary structure of genomic (nuclear) and mtDNA in human embryos using a microarray according to claim 1, wherein said step of DNA amplification comprises amplifying DNA from a single blastomere or trophectoderm cell(s) from said viable embryo by a multiple displacement amplification reaction.

6. The method for interrogating content and primary structure of genomic (nuclear) and mtDNA in human embryos using a microarray according to claim 5, further comprising purifying the amplified DNA.

7. The method for interrogating content and primary structure of genomic (nuclear) and mtDNA in human embryos using a microarray according to claim 1, wherein said step of performing pre-implantation genetic diagnosis and screening further comprises the substeps of:
assaying the purified/amplified DNA by high-density microarray interrogation of naturally occurring DNA sequences to yield a genomic dataset,
interrogating the genomic dataset and generating a high-resolution map, and
grading embryos to determine a selected subset.

8. The method for interrogating content and primary structure of genomic (nuclear) and mtDNA in human embryos using a microarray according to claim 7, further comprising a substep of using fluorescence in situ Hybridization (FISH) to identify chromosome aberrations.

9. The method for interrogating content and primary structure of genomic (nuclear) and mtDNA in human embryos using a microarray according to claim 1, comprising a final step of transferring a selected subset of graded embryos to the patient.

10. A method for isolating and amplifying DNA from single cells to run a modified microarray platform in:
fertilizing human ova in vitro with human sperm under sterile laboratory conditions;
growing said fertilized human ova (zygotes) in an embryo culture for 2-8 days;
on said third day, assessing said zygotes with a microscope and grading based on pre-defined morphological criteria;
biopsying viable embryos;
transferring a single blastomere or trophectoderm cell(s) from said viable embryo for genetic analysis;
amplifying DNA from said single blastomere or trophectoderm cell(s) from said viable embryo by an optimized multiple displacement amplification reaction;
purifying the amplified DNA;
assaying the purified/amplified DNA by high-density microarray interrogation of naturally occurring DNA sequences to yield a genomic dataset;
interrogating the genomic dataset and generating a high-resolution map;
grading embryos to determine a selected subset;
transferring the selected subset of graded embryos to patient.

11. The method for isolating and amplifying DNA from single cells according to claim 10, wherein said step of assessing said zygotes with a microscope and grading based on pre-defined morphological criteria further comprises a visual inspection of embryo culture via microscopy.

12. The method for isolating and amplifying DNA from single cells according to claim 11, wherein said step of assessing said zygotes with a microscope and grading embryo cleavage includes ≧2 cells of cleaving embryos.

13. A system for genetic testing of human embryos, comprising:
an embryo grading system for grading IVF embryos based on pre-defined morphological criteria;
an embryo biopsy procedure;
a DNA extraction procedure for extracting DNA from a biopsy sample;
a DNA amplification protocol for single cell analyses;
a single-cell microarray for identifying genotype/haplotype patterns and for prescreening genetic diseases and predictors; and
a transfer procedure for transferring high-grade embryos to a patient.

14. The system for genetic testing of human embryos, wherein said embryo biopsy technique is minimally invasive to reduce exogenous cellular contamination.

15. The system for genetic testing of human embryos, wherein said DNA amplification protocol for single cell analyses comprises an optimized multiple displacement amplification reaction prior to whole genome analysis.

16. The system for genetic testing of human embryos, wherein said microarray comprises a high-density interrogation of DNA sequences.
